(19) United States
(12) Patent Application Publication
(10) Pub. No.: US 2011/0059028 A1
(43) Pub. Date: Mar. 10, 2011

(54) METHOD FOR MEMBRANE ELECTROLYSIS AND THE USE OF THE PRODUCTS OBTAINED BY IT

(76) Inventor: Burkhard Ponitz, Dillendorf (DE)

(21) Appl. No.: 12/452,714

(22) PCT Filed: Jul. 5, 2008
(86) PCT No.: PCT/EP2008/005501

§ 371 Date: Aug. 25, 2010

(30) Foreign Application Priority Data
Jul. 18, 2007 (DE) 10 2007 033 445.3

(51) Int. Cl. A61K 8/19; C25B 1/00; C25B 3/00; B08B 9/08; B08B 9/027; A23K 1/18; C02F 1/52; B08B 3/08; B09C 1/08; A61K 33/14; A61Q 11/00; A61P 17/02; A61P 1/00; A61P 31/10; A61P 31/00; A61P 17/00

(52) U.S. Cl. 424/49; 205/334; 134/42; 134/22.13; 426/2; 210/702; 210/665; 405/128.75; 424/677

(57) ABSTRACT

A metal sulphate is added to the alkali metal chloride solution in membrane electrolysis in order to increase the amount of catholyte solution formed. At the same time, a metal sulphate is added to the anolyte solution in order to improve its stability.

The present invention relates to a method of membrane electrolysis, as defined in the pre-characterising part of Claim 1, as well as to the use of anolyte and catholyte solutions obtained by the said method.

Membrane electrolysis is a known process used in water purification (see e.g. EP 1380543 A1, EP 1382573 A1 and WO 2006/040709 A1). It is carried out with an electrolytic cell comprising an anode space that houses the anode, and a cathode space that houses the cathode and which is separated from the anode space by a membrane.

An aqueous solution of sodium chloride is introduced into the anode space and the cathode space as the electrolyte. As a result, the anions and other negatively charged particles in the electrolyte migrate towards the anode, while the cations and similar positively charged particles migrate towards the cathode. The negatively charged particles present in the cathode space, and the positively charged particles present in the anode space must therefore pass through the membrane in order to reach the anode and cathode, respectively.

This leads to the formation of an anolyte solution in the anode space, which solution has a positive redox potential and therefore possesses oxidizing properties. The catholyte solution formed in the cathode space has a negative redox potential and therefore possesses reducing properties. In addition, the catholyte solution has strongly basic characteristics, while the anolyte solution is acidic.

The anolyte solution that contains sodium hypochlorite, hydrogen peroxide, chlorine and ozone as metastable compounds is introduced into the water to be purified, while the catholyte solution, which has so far been formed in much smaller amounts than the anolyte solution is either discarded or used for the precipitation of heavy metals (see EP 1380543 A1).

The task of the present invention is to open up new fields of application for both the catholyte solution and the anolyte solution.

The present invention therefore provides, on the one hand, a method that makes it possible to obtain the catholyte solution in a larger amount, and on the other hand, a method by which the metastable oxidizing properties of the anolyte solution can be stabilized, so it can be used in other fields as well.

It has now been found surprisingly that catholyte formation can be greatly increased if the sodium chloride solution that is used as the electrolyte contains a metal sulphate. It is possible that this accelerates the migration of the cations from the anode space to the cathode space across the membrane. Since a large amount of catholyte is now formed, the invention opens up numerous possible new applications for the catholyte solution.

The method according to the invention can be carried out for example with the aid of a commercially available device that is used for membrane electrolysis in water purification, and in which the flow regulator on the cathode side is modified in order to increase the amount of catholyte flowing through.

In addition to the raised catholyte formation, it has also been found surprisingly that the addition of a metal sulphate to the electrolyte also leads to the formation of other highly active metastable compounds from the sulphate anions in the anolyte solution.

The electrolyte preferably consists of sodium chloride, but it can also be formed by another alkali metal chloride. For example, a mixture of sodium chloride and potassium chloride has been found suitable in particular, where the potassium chloride can represent up to 50 wt-% of the total weight of the alkali metal chlorides. The alkali metal solution is preferably removed from its storage vessel in the form of a saturated solution, and then diluted with the required amount of water, e.g. in a ratio by volume of between 1:5 and 1:20. The sodium sulphate is preferably added to the saturated alkali metal chloride solution while it is still in the storage vessel. The latter can be heated in order to keep the metal sulphate in solution. The metal sulphate is preferably added to the alkali metal chloride solution in an amount of 1-50 g per litre and especially 5-20 g per litre.

The water used to dilute the alkali metal chloride solution preferably contains little or no carbonate, i.e. it preferably has a German degree of hardness of 5 at most, which means that its calcium carbonate content does not exceed 50 mg/l.

The metal sulphate added to the alkali metal chloride solution is preferably an alkali metal sulphate, especially sodium sulphate, or it is an alkaline earth metal sulphate, especially magnesium sulphate.

The anolyte solution can have a redox potential of over +1200 mV and a pH of 1-3, while the catholyte solution can have a redox potential of −600 to −1000 mV and a pH of more than 12.

The second aim of the invention, i.e. to provide a stable anolyte solution in order to open up new fields of application for the anolyte solution, is preferably achieved by adding a metal sulphate to the anolyte solution. The metal sulphate can be added in an amount of 0.1-20 g, especially 0.5-5 g and more preferably 1-2 g per litre of anolyte solution.

It has now been found surprisingly that this stabilizes the metastable compounds in the anolyte solution and therefore reliably extends their activity for months, so that the anolyte can be assumed to remain stable in storage for 1-2 years.

The metal sulphate used to stabilize the anolyte solution is preferably aluminium sulphate, especially in the form of potassium alum.

Potassium alum, i.e. potassium aluminium sulphate, has long been known as an active substance of natural medicines. It is used e.g. in the case of gastric, intestinal and bladder weakness, but also in the case of haemorrhages. Alum is known to disinfect the mucous membranes and wounds. However, it also acts as a desiccant, partial coagulant and secretion inhibitor, so its medical use is limited.

It has now been found surprisingly that an anolyte solution that contains alum exerts a remarkable cleansing, decontaminating and disinfecting effect in the oral and pharyngeal region without exerting any deleterious effects on the teeth, dental fillings and tongue, and also without having any desiccant and secretion inhibiting action. The alum-containing anolyte solution according to the invention is therefore very suitable for use as a mouthwash. For example, it can be used to treat refractory inflammations in the oral and pharyngeal region. It stops the bleeding e.g. during dental treatment within a few minutes. The mouthwash can also be used prophylactically in order to prevent periodontal disorders, gingivitis and stomatitis.

The mouthwash can also contain magnesium sulphate, which further improves its stability in storage. The anolyte solution can contain for example 0.5-10 g and especially 1-2 g of magnesium sulphate per litre of solution.

The mouthwash can also contain other additives that are in general use, for example flavouring substances, sweeteners, essential oils and suchlike.

It has been found that the mouthwash according to the invention reliably eliminates the microorganisms that are responsible for plaque, gingivitis and periodontitis. As a result, it efficiently prevents infections and ensures that any wounds that may be present heal quickly. At the same time, it does not cause any discoloration of the gums, teeth and tongue.

However, the anolyte solution according to the invention can be used not only as a mouthwash, but also for a great variety of other indications, both in human medicine and in veterinary practice. For example, it has a strongly biocidal—especially antibacterial and fungicidal—effect.

Thus, the anolyte solution according to the invention can be used for the treatment of wounds, e.g. in the case of cutaneous lesions, as well as for the treatment of ulcers. The anolyte solution according to the invention has proved excellent especially for the treatment of the gastrointestinal tract, e.g. for the elimination of disorders and haemorrhages caused by Helicobacter or Candida species. In addition to its powerful antibacterial action, the anolyte solution according to the invention also exerts a strong fungicidal effect. It can successfully combat fungal infections both in human beings (e.g. onychomycosis), and e.g. hoof rot, “scratches” and saddle fungi in horses.

In addition, the anolyte solution according to the invention can be used to treat eczema, especially summer eczema in horses, and also frontal sinus suppuration e.g. in horses, as well as inflammations and infections in man and animals, including prophylaxis against ticks. The anolyte solution according to the invention can also be used especially in the case of disorders that can no longer be treated because of resistance to antibiotics.

Another field of application for the anolyte solution according to the invention is the improvement of the shelf life of creams, for example those used for skin care. This is because the anolyte solution according to the invention does not only have a disinfecting action but also a strong emulsifying effect. In addition, it has the advantage over other additives used in creams that it does not cause allergies.

It has also been found that the anolyte solution according to the invention makes an excellent additive to the drinking water used in poultry farming.

For example, last year, when chickens had to be kept in their houses due to bird flu, the anolyte solution according to the invention was given to half the birds on a poultry farm with a stock of several hundred chickens in their drinking water at a dilution of 1 part by volume of anolyte solution to 10 parts by volume of water in the first week and then at a dilution of 1:100 in the next five weeks. The other half of the stock was not given any anolyte solution in their drinking water and served as the controls. After six weeks, the weight increase of the chickens receiving the anolyte solution was more than 30% higher than that of the chickens in the control group.

In addition to its use in poultry farming, the anolyte solution is also suitable for use as a drinking water additive in animal husbandry in general.

Furthermore, the anolyte solution according to the invention is very suitable for use as a cleaning agent e.g. in the food sector. For example, it can be used to clean pipes, containers and suchlike, e.g. in breweries and dairy establishments, or quite generally to clean plants and equipment used in the production of food and drinks, including in particular the production of ice for cooling or chilling purposes. Thus, ice made in plants cleaned with the anolyte solution according to the invention can be very successfully brought into direct contact with easily perishable food products like fish, since it is free of microorganisms.

This means that the anolyte solution produced according to the invention is of general applicability when it comes to cleaning surfaces in the food and drink sector. It can be used especially for cleaning plants, equipment and objects involved in food production or in the manufacture of processing aids like ice for cooling or chilling, which come into contact with food.

A combination of the anolyte solution according to the invention and the catholyte solution according to the invention can be used here in particular. This applies not only to the food sector, but also to other cleaning applications. In such a case, first the strongly basic catholyte solution according to the invention is used for cleaning, followed by the anolyte solution according to the invention.

The catholyte solution according to the invention is also very suitable for sterilizing and disinfecting surfaces. For example, the catholyte solution according to the invention can be used for cleaning surfaces in doctors' surgeries, hospitals and similar health institutions, or in old people's homes. Owing to the high pH of the catholyte solution, the unpleasant smell often typical of these establishments is also eliminated here.

Surface treatment with the catholyte solution can be followed by treatment with the anolyte solution according to the invention in order to complete the sterilization and disinfection. The anolyte solution according to the invention has a strong cleaning action and can therefore be used for the production of cleaning agents.

Furthermore, the catholyte solution according to the invention can be used for example to neutralize the pH of acidic soils, owing to its high alkalinity. For example, fungal growth in the soil can be suppressed by shifting the pH from acidic values to the neutral region, thereby noticeably improving the growth of plants, due to an increased uptake of nutrients.

Another field of application for the catholyte solution according to the invention is the purification of water that is strongly contaminated with heavy metals, for example in textile dyeing plants, in the case of water for fire-fighting purposes, and with the effluent formed in cold-rolling mills. For example, when the pH of the water is raised to 9-10 by the catholyte solution according to the invention, the heavy metals are precipitated out virtually quantitatively. The floccules formed can be bound in a mineral powder, such as basalt dust for example, and separated off e.g. by filtration on a sintered metal filter. The residual water formed by the filtrate can be further purified for example with the aid of the anolyte solution according to the invention.

1. Membrane electrolysis method, whereby an aqueous alkali metal chloride solution is introduced into both the anode space and the cathode space [of] an electrolytic cell, which are separated from each other by a membrane, in order to form an anolyte solution and a catholyte solution, characterised in that a metal sulphate is added to the said alkali metal chloride solution to increase the catholyte production.

2. Method according to claim 1, characterised in that 1 to 50 grams of metal sulphate per litre are added to the said alkali metal chloride solution.

3. Method according to claim 1, characterised in that the metal sulphate is an alkali metal sulphate and/or alkaline earth metal sulphate.

4. Method according to claim 3, characterised in that the alkali Metal sulphate is sodium sulphate or that the alkaline earth metal sulphate is magnesium sulphate.

5. Method according to claim 1, characterised in that the aqueous alkali metal chloride solution thus introduced contains less than 50 mg of calcium carbonate per litre.

6. Membrane electrolysis method, in particular according to claim 1, characterised in that a metal sulphate is added to the anolyte solution.

7. Method according to claim 6, characterised in that the amount of metal sulphate added is 0.1 to 20 grams per litre.

8. Method according to claim 6, characterised in that the metal sulphate is aluminium sulphate or magnesium sulphate.

9. Method according to claim 8, characterised in that alum is used as the aluminium sulphate.

10. Application in human or veterinary medicine of the anolyte solution obtained according to claim 1.

11. Use according to claim 10, characterised in that the anolyte solution is used for oral and/or dental hygiene.

12. Use according to claim 11, characterised in that the anolyte solution is used for the prevention and/or treatment of periodontal disorders, gingivitis and/or stomatitis.

13. Use according to claim 11, characterised in that the anolyte solution is used as a mouthwash.

14. Use according to claim 10, characterised in that the anolyte solution is used for treating wounds.

15. Use according to claim 10, characterised in that the anolyte solution is used for treating the gastrointestinal tract.

16. Use according to claim 10, characterised in that the anolyte solution is used for the prevention and/or treatment of fungal diseases.

17. Use according to claim 10, characterised in that the anolyte solution is used for treating eczema and/or infections.

18. Application of the anolyte solution obtained according to claim 1 for extending the shelf-life of creams.

19. Application of the anolyte solution obtained according to claim 1 as an additive to drinking water in poultry farming or animal husbandry.

20. Application of the anolyte solution obtained according to claim 1 as a cleaning agent in the food industry.

21. Application of the catholyte solution obtained according to claim 1 for sterilization and/or disinfection of surfaces.

22. Use according to claim 21, characterised in that after disinfection of the surfaces by means of the catholyte solution, the anolyte solution is then used.

23. Application of the catholyte solution obtained according to claim 1 for treating the soil.

24. Application of the catholyte solution according to claim 1 to treat water polluted with heavy metals by precipitating out the heavy metals.

25. Use according to claim 24, characterised in that the heavy metals precipitated out by means of the catholyte solution are bound using a powdered mineral and then separated off.
